(19) United States
(12) Patent Application Publication
(10) Pub. No.: US 2025/0166804 A1
(43) Pub. Date: May 22, 2025

(54) INFORMATION INTERACTION METHOD, PLATFORM, DEVICE, AND NON-TRANSITORY COMPUTER STORAGE MEDIUM

(71) Applicant: BOE Technology Group Co., Ltd., Beijing (CN)

(72) Inventors: Fang ZHAI, Beijing (CN); Shuobin LIANG, Beijing (CN); Hongliang WANG, Beijing (CN); Yanyang HU, Beijing (CN); Can CUI, Beijing (CN)

(21) Appl. No.: 19/026,257

(22) Filed: Jan. 16, 2025

Related U.S. Application Data
(63) Continuation of application No. PCT/CN2023/084510, filed on Mar. 28, 2023.

(51) Int. Cl. G16H 40/20; G16H 10/60

(52) U.S. Cl. CPC G16H 40/20; G16H 10/60

(57) ABSTRACT

Provided is an information interaction method, applicable to an information interaction platform. The method includes: acquiring patient description information provided by a terminal corresponding to a first institution, wherein the first institution is an institution registered on the information interaction platform, and the terminal corresponding to the first institution is configured to acquire the patient description information provided by a target patient of the first institution; determining role data of a plurality of medical practitioners corresponding to the first institution on the information interaction platform, wherein the role data is configured to identify information of the medical practitioners in the first institution; and transmitting, based on the role data, the patient description information to a terminal of at least one medical practitioner of the plurality of medical practitioners corresponding to the first institution.

### CROSS-REFERENCE TO RELATED APPLICATION

The application is a continuation application of international application No. PCT/CN2023/084510, filed on Mar. 28, 2023, the content of which is herein incorporated by reference in its entirety.

### TECHNICAL FIELD

The present disclosure relates to the field of information interaction technologies, and in particular, relates to an information interaction method, a platform, a device and a non-transitory computer storage medium.

### BACKGROUND

Currently, several web platforms can provide medical services from a plurality of medical institutions to patients, and patients can interact with their doctors for information on these web platforms.

### SUMMARY

Embodiments of the present disclosure provide an information interaction method, a platform, a device, and a non-transitory computer storage medium. The technical solutions are as follows.

According to some embodiments of the present disclosure, an information interaction method, applied in an information interaction platform, is provided. The method includes:
acquiring patient description information provided by a terminal corresponding to a first institution, wherein the first institution is an institution registered on the information interaction platform, and the terminal corresponding to the first institution is configured to acquire the patient description information provided by a target patient of the first institution;
determining role data of a plurality of medical practitioners corresponding to the first institution on the information interaction platform, wherein the role data is configured to identify information of the medical practitioners in the first institution; and
transmitting, based on the role data, the patient description information to a terminal of at least one medical practitioner of the plurality of medical practitioners corresponding to the first institution.

In some embodiments, prior to acquiring the patient description information provided by the terminal corresponding to the first institution, the method further includes:
acquiring first registration information provided by a terminal of a first medical practitioner for the first institution;
performing verification on the first registration information;
in response to the first registration information passing the verification, establishing first role data of the first medical practitioner in the first institution;
acquiring second registration information provided by the terminal of the first medical practitioner for a second institution;
performing verification on the second registration information; and
in response to the second registration information passing the verification, establishing second role data of the first medical practitioner in the second institution.

In some embodiments, performing the verification on the first registration information includes:
providing the first registration information to the first institution, wherein the first institution is configured to perform the verification on the first registration information; and
in response to receiving verification pass information provided by the first institution, determining that the first registration information passes the verification.

In some embodiments, the information interaction platform further includes: a certificate issuer node, a first user node, and a first institution node corresponding to the first institution in a block chain; and
acquiring the first registration information provided by the terminal of the first medical practitioner for the first institution includes:
acquiring, by the first user node, the first registration information provided by the terminal of the first medical practitioner for the first institution;
providing the first registration information to the first institution includes:
providing the first registration information to the first institution node; and
in response to receiving the verification pass information provided by the first institution, determining that the first registration information passes the verification includes:
in response to acquiring the verification pass information, providing a certificate to the first user node by the certificate issuer node, wherein the certificate is configured to determine that the first registration information passes the verification.

In some embodiments, upon acquiring the patient description information provided by the terminal corresponding to the first institution, the method further includes:
storing the patient description information provided by the terminal corresponding to the first institution in the first institution node;
acquiring patient description information provided by a terminal corresponding to the second institution; and
storing the patient description information provided by the terminal corresponding to the second institution in the second institution node.

In some embodiments, the role data includes a data query range of the medical practitioners in the first institution; and the method further includes:
acquiring, by the first institution node, a query request provided by the first user node, wherein the query request is configured to acquire the patient description information of the target patient of the first institution, and the first institution node is configured to determine, based on the role data, whether the patient description information is within a query range of the first medical practitioner; and
in response to determining that the patient description information is within the query range of the first medical practitioner, providing the patient description information to the first user node.

In some embodiments, acquiring, by the first institution node, the query request provided by the first user node includes:
receiving, by the first institution node, the query request transmitted by a to-be-verified node;
performing identity authentication on the to-be-verified node; and
in response to the to-be-verified node passing the identity authentication, determining the to-be-verified node to be the first user node.

In some embodiments, performing the identity authentication on the to-be-verified node includes:
performing verification, by the first institution node, on a certificate provided by the to-be-verified node;
in response to the certificate provided by the to-be-verified node passing the verification, determining that the to-be-verified node passes the identity authentication.

In some embodiments, transmitting, based on the role data, the patient description information to the terminal of at least one medical practitioner of the plurality of medical practitioners corresponding to the first institution includes:
determining, based on the role data, whether the target patient has a corresponding medical practitioner in the first institution; and
in response to the target patient having the corresponding medical practitioner in the first institution, transmitting the patient description information to a terminal of the corresponding medical practitioners.

In some embodiments, prior to transmitting, based on the role data, the patient description information to the terminal of at least one medical practitioner of the plurality of medical practitioners corresponding to the first institution, the method further includes:
acquiring authorization information provided by the target patient, wherein the authorization information is configured to authorize the information interaction platform, such that the information interaction platform is capable of transmitting the patient description information to the terminal of at least one medical practitioner of the plurality of medical practitioners corresponding to the first institution.

According to some embodiments of the present disclosure, an information interaction platform is provided. The information interaction platform is configured to:
acquire patient description information provided by a terminal corresponding to a first institution, wherein the first institution is an institution registered on the information interaction platform, and the terminal corresponding to the first institution is configured to acquire the patient description information provided by a target patient of the first institution;
determine role data of a plurality of medical practitioners corresponding to the first institution on the information interaction platform, wherein the role data is configured to identify information of the medical practitioners in the first institution; and
transmit, based on the role data, the patient description information to a terminal of at least one medical practitioner of the plurality of medical practitioners corresponding to the first institution.

In some embodiments, the information interaction platform includes a terminal of a first medical practitioner, wherein the terminal of the first medical practitioner is configured to acquire first registration information for the first institution; and
the information interaction platform is further configured to:
perform verification on the first registration information; and
in response to the first registration information passing the verification, establish first role data of the first medical practitioner in the first institution;
the terminal of the first medical practitioner is further configured to acquire second registration information for a second institution; and
the information interaction platform is further configured to:
perform verification on the second registration information; and
in response to the second registration information passes the verification, establish second role data of the first medical practitioner in the second institution.

In some embodiments, the information interaction platform further includes a certificate issuer node, a first user node, and a first institution node corresponding to the first institution in a block chain;
the first user node is configured to acquiring the first registration information for the first institution;
the first institution node is configured to perform verification on the first registration information; and
the certificate issuer node is configured to provide a certificate to the first user node in response to the first registration passing the verification, wherein the certificate is configured to determine that the first registration information passes the verification.

According to some embodiments of the present disclosure, an information interaction device is provided. The device includes a processor and a memory, the memory storing at least one instruction, at least one program, a code set, or an instruction set therein, wherein the processor, when loading ad executing the at least one instruction, the at least one program, the code set, or the instruction set, is caused to perform the information interaction method as described above.

According to some embodiments of the present disclosure, a non-transitory computer storage medium is provided. The non-transitory computer storage medium stores at least one instruction, at least one program, a code set, or an instruction set therein, wherein the at least one instruction, the at least one program, the code set, or the instruction set, when loaded and executed by a processor, causes the processor to perform the information interaction method as described above.

According to some embodiments of the present disclosure, a computer program product or a computer program is provided. The computer program product or computer program has at least one computer instruction stored in a computer-readable storage medium. The at least one computer instruction, when read and loaded by a processor of a computer device, causes the computer device to perform the information interaction method as described above.

According to some embodiments of the present disclosure, an information interaction apparatus is provided. The information interaction apparatus is applicable to an information interaction platform. The information interaction apparatus includes:
a first acquiring module, configured to acquire patient description information provided by a terminal corresponding to a first institution, wherein the first institution is an institution registered on the information interaction platform, and the terminal corresponding to the first institution is configured to acquire patient description information provided by a target patient of the first institution;
a role data determining module, configured to determine role data of a plurality of medical practitioners corresponding to the first institution on the information interaction platform, wherein the role data is configured to identify information of the medical practitioners in the first institution; and
a description information transmitting module, configured to transmit, based on the role data, the patient description information to a terminal of at least one medical practitioner of the plurality of medical practitioners corresponding to the first institution.

In some embodiments, the information interaction apparatus further includes:
a first registering module, configured to acquire first registration information provided by a terminal of a first medical practitioner for the first institution;
a first verifying module, configured to perform verification on the first registration information;
a first establishing module, configured to establish first role data of the first medical practitioner in the first institution in response to the first registration information passing the verification;
a second registering module, configured to acquire second registration information provided by the terminal of the first medical practitioner for a second institution;
a second verifying module, configured to perform verification on the second registration information; and
a second establishing module, configured to establish second role data of the first medical practitioner in the second institution in response the second registration information passing the verification.

In some embodiments, the first verifying module includes:
a first transmitting unit, configured to transmit the first registration information to a verification terminal, wherein the verification terminal is configured to perform verification on the first registration information; and
a first determining unit, configured to determine that the first registration information passes the verification in response to receiving verification pass information provided by the verification terminal.

In some embodiments, the information interaction platform further includes a certificate issuer node, a first user node, and a first institution node corresponding to the first institution in a block chain.

The first registration module is further configured to:
acquire the first registration information provided by the first user node for the first institution.

The first determining unit is further configured to:
provide a certificate to the first user node by the certificate issuer node in response to receiving the verification pass information provided by the first institution node, wherein the certificate is configured to determine that the first registration information passes the verification.

In some embodiments, upon acquiring the patient description information provided by the terminal corresponding to the first institution, the information interaction unit further includes:
a first storing module, configured to store the patient description information provided by the terminal corresponding to the first institution in the first institution node;
a second acquiring module, configured to acquire patient description information provided by a terminal corresponding to a second institution; and
a second storing module, configured to store the patient description information provided by the terminal corresponding to the second institution in the second institution node.

In some embodiments, the role data incudes a data query range of the medical practitioners in the first institution, and the information interaction apparatus further includes:
a request acquiring module, configured to acquire, by the first institution node, a query request provided by the first user node, wherein the query request is configured to acquire the patient description information of the target patient in the first institution; and the first institution node is configured to determine, based on the role data, whether the patient description information is within a query range of the first medical practitioner; and
a query information transmitting module, configured to transmit the patient description information to the first user node in response to determining that the patient description information is within the query range of the first medical practitioner.

In some embodiments, the request acquiring module includes:
a request receiving unit, configured to receive a query request transmitted by a to-be-verified node by the first institution node;
a verifying unit, configured to perform identity authentication on the node to-be-verified; and
a determining unit, configured to determine, in response to the to-be-verified node passing the identity authentication, that the to-be-verified node is the first user node.

In some embodiments, the verifying unit is further configured to:
perform verification on a certificate provided by the to-be-verified node; and
determine that the to-be-verified node passes the identity authentication in response to the certificate provided by the to-be-verified node passing the verification.

In some embodiments, the description information transmitting module is further configured to:
determine, based on the role data, whether the target patient has a corresponding medical practitioner in the first institution; and
in response to the target patient having the corresponding medical practitioner in the first institution, transmit the patient description information to a terminal of the corresponding medical practitioner.

In some embodiments, the information interaction apparatus further includes:
an authorizing module, configured to acquire authorization information provided by the target patient, wherein the authorization information is configured to authorize the information interaction platform, such that the information interaction platform is capable of transmitting the patient description information to the terminal of at least one of the plurality of medical practitioners corresponding to the first institution.

### BRIEF DESCRIPTION OF DRAWINGS

For clearer descriptions of the technical solutions in the embodiments of the present disclosure, the following briefly introduces the accompanying drawings to be required in the descriptions of the embodiments. Apparently, the accompanying drawings in the following description show merely some embodiments of the present disclosure, and persons of ordinary skills in the art may still derive other drawings from these accompanying drawings without creative efforts.

FIG. 1 is a schematic diagram of an implementation environment according to some embodiments of the present disclosure;

FIG. 2 is a schematic diagram of another implementation environment according to some embodiments of the present disclosure;

FIG. 3 is a flowchart of an information interaction method according to some embodiments of the present disclosure;

FIG. 4 is a flowchart of another information interaction method according to some embodiments of the present disclosure;

FIG. 5 is a schematic diagram of medical practitioners, institution, and role data according to some embodiments of the present disclosure;

FIG. 6 is another schematic diagram of medical practitioners, institution, and role data according to some embodiments of the present disclosure;

FIG. 7 is a schematic diagram of some application scenarios of an information interaction method according to some embodiments of the present disclosure;

FIG. 8 is a flowchart of still another information interaction method according to some embodiments of the present disclosure;

FIG. 9 is a schematic diagram of permission management according to some embodiments of the present disclosure; and

FIG. 10 is a block diagram of an information interaction apparatus according to some embodiments of the present disclosure.

Definite embodiments of the present disclosure, shown by the above accompanying drawings, will be described in further detail hereinafter. These accompanying drawings and textual descriptions are not intended to limit the scope of the concepts of the present disclosure in any form, but rather to illustrate the concepts of the present disclosure for those skilled in the art by reference to particular embodiments.

### DETAILED DESCRIPTION

The present disclosure is described in further detail with reference to the accompanying drawings, to clearly present the objects, technical solutions, and advantages of the present disclosure.

In an information interaction method, the patient provides description information about himself to the platform, and the platform analyses the description information and the doctors from the plurality of institutions registered on the platform, and transmits the description information about the patient to a plurality of matched doctors, such that the doctors are capable of viewing the description information.

However, the above methods are less protective of the patient's privacy.

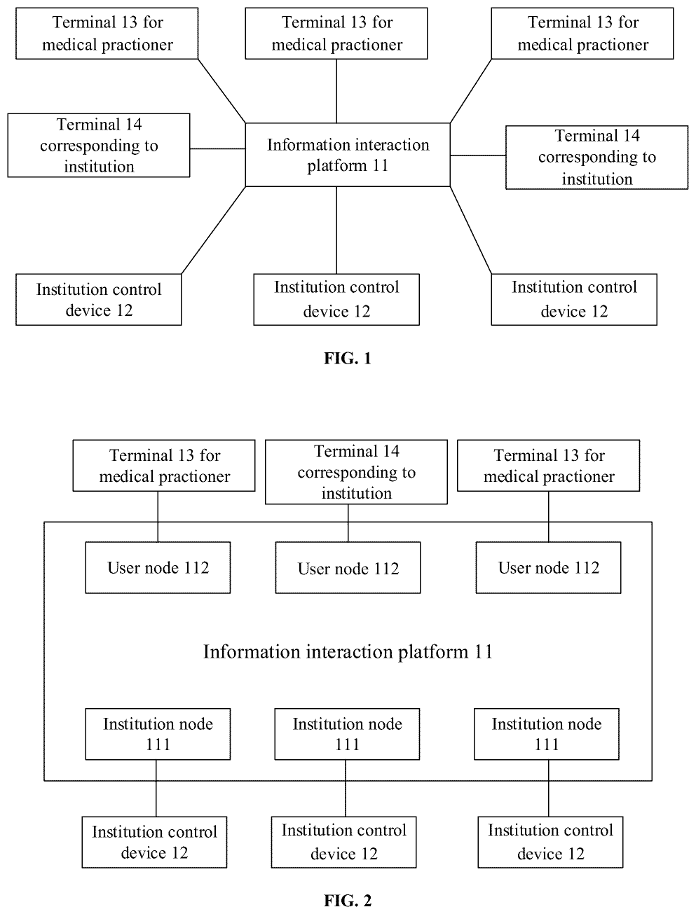

FIG. 1 is a schematic diagram of an implementation environment according to some embodiments of the present disclosure. The implementation environment includes an information interaction platform 11, a plurality of institution control devices 12, a plurality of terminals 13 for medical practitioners, and a plurality of terminals 14 corresponding to institutions.

The information interaction platform 11 has a wired or wireless connection with the plurality of institutions 12, the plurality of terminals 13 for medical practitioners, and the plurality of first terminals 14 corresponding to a first institution, such that platform 11 is capable of acquiring information provided by the plurality of institutions 12, the plurality of terminals 13 for medical practitioners, and the plurality of terminals 14 corresponding to the institution, and transmitting information to the plurality of institutions 12, the plurality of terminals 13 for medical practitioners, and the plurality of terminals 14 corresponding to the first institution. The information interaction platform 11 includes a server, a cluster of servers, or a cloud server.

The institution control devices 12 include management terminals for each institution, which include public health institutions (governments at all levels, health administrative agencies at all levels, disease control agencies, family planning agencies, and health supervision agencies.), hospitals, and health management institutions. Each institution corresponds to a plurality of terminals 14. The terminals 14 include a variety of terminals corresponding to the institution 12, such as a variety of medical devices and intelligent terminals. The medical device collects patient description information of the patient and, under the authorization of the patient, provides the patient description information to the information interaction platform 11, while the intelligent terminal acquires the patient description information input by the patient and, under the authorization of the patient, provides the patient description information to the information interaction platform 11. In addition, the institution control device 12 of each institution is capable of managing the terminals 14 corresponding to the institution.

The terminals 13 for medical practitioners include terminals used by medical practitioners at each institution, through which the medical practitioners at each institution is capable of acquiring the patient depiction information and transmitting information to the patient, the information interaction platform 11, and the institution.

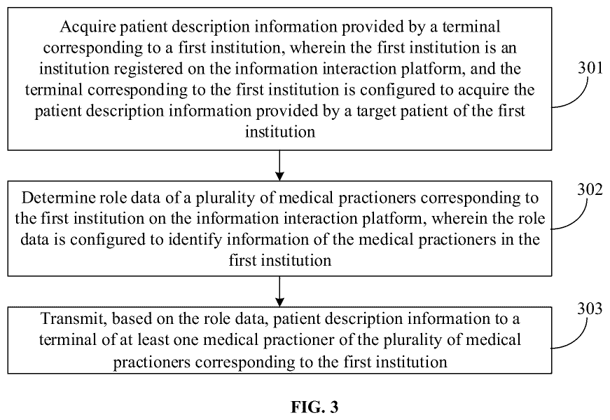

FIG. 2 is a schematic diagram of another implementation environment according to some embodiments of the present disclosure. The implementation environment includes an information interaction platform 11, a plurality of institutional control devices 12, a plurality of terminals 13 for medical practitioners, and a plurality of terminals 14 corresponding to institutions.

The information interaction platform 11 is a block chain platform, and the corresponding information interaction platform 11 includes a plurality of nodes in the block chain, through which the information interaction platform 11 stores data and interacts with other terminals for information. For example, the information interaction platform 11 includes a plurality of institution nodes 111 and a plurality of user nodes 112. The plurality of institution nodes 111 correspond to the plurality of institutional control devices 12, and the institutional control devices 12 perform information interaction in the information interaction platform 11 through the institution nodes. The plurality of user nodes 112 correspond to the plurality of terminals 13 for medical practitioners and the plurality of terminals 14 corresponding to institutions, and the terminals 13 for medical practitioners interact with the terminals 14 corresponding to institutions for information in the information interaction platform 11 through the user nodes 112.

In addition, the information interaction platform 11 includes some other nodes, such as certificate issuer nodes 113, which are configured to issue certificates to other nodes to prove identities.

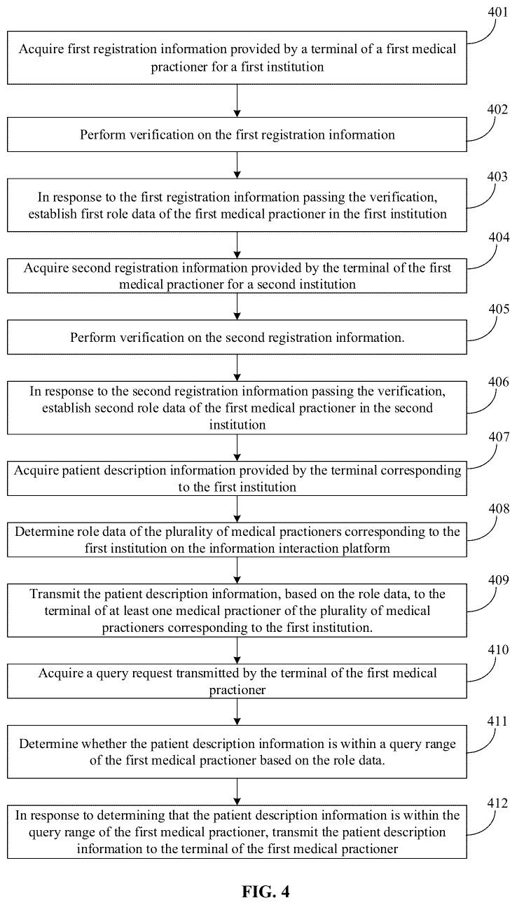

FIG. 3 is a flowchart of an information interaction method according to some embodiments of the present disclosure. The method is applicable to the information interaction platform in the implementation environment shown in FIG. 1 or FIG. 2. The method includes the following steps.

In step 301, patient description information provided by a terminal corresponding to a first institution is acquired, wherein the first institution is an institution registered on the information interaction platform, and the terminal corresponding to the first institution is configured to acquire the patient description information provided by a target patient of the first institution.

In step 302, role data of a plurality of medical practitioners corresponding to the first institution on the information interaction platform is acquired, wherein the role data is configured to identify information of the medical practitioners in the first institution.

In step 303, the patient description information is transmitted, based on the role data, to a terminal of at least one medical practitioner of the plurality of medical practitioners corresponding to the first institution.

In summary, according to the information interaction method provided by the embodiments of the present disclosure, by corresponding the patient to the institution in the information interaction platform, upon acquiring the patient description information provided by the terminal corresponding to the first institution, the patient description information is transmitted to the terminal of the medical practitioners of the first institution, based on the role data of the plurality of medical practitioners corresponding to the first institution. In this way, the information interaction platform information is capable of transmitting the patient description information of the patient corresponding to each institution to the medical practitioners of the corresponding institution, and the medical practitioners from different institutions cannot see the patient description information of other institutions, such that the privacy of the patient description information is improved, which solves the problem of low protection of the privacy of the patients in the related art, and thus the effect of improving the protection of the privacy of the patients is achieved.

The method according to the embodiments of the present disclosure is used to achieve multi-point practice of physicians. The multi-point practice of physicians refers to the act of a qualified practicing physician being employed to practice in more than two medical institutions after being registered by a health administrative department. The multi-point practice of physicians is conducive to balancing medical resources in various places, to the improvement of the level of physicians in communities, primary healthcare institutions, and underdeveloped areas, and to alleviating the healthcare needs of the grassroots. At the same time, a rational flow of medical resources is achieved.

In some embodiments of the present disclosure, a plurality of institutions are registered on the information interaction platform, and the medical practitioners are registered on the information interaction platform as the medical practitioners of at least one of the plurality of institutions. Further, the medical practitioners have different roles in different institutions. For example, a medical practitioner is a doctor in Institution A and is a health manager in Institution B.

FIG. 4 is a flowchart of another information interaction method according to some embodiments of the present disclosure. The method is applicable to the information interaction platform in the implementation environment shown in FIG. 1. The method includes the following steps.

In step 401, first registration information provided by a terminal of a first medical practitioner for a first institution is acquired.

The information interaction platform has a plurality of registered institutions, and the information interaction platform accepts the registration of medical practitioners for the institutions. and each of the medical practitioners is capable of registering for a plurality of institutions.

Exemplarily, in applying the information interaction method according to some embodiments of the present disclosure, the information interaction platform acquires the first registration information provided by the terminal of the first medical practitioner for the first institution. The first medical practitioner is a licensed physician who is eligible and registered in the health administration department. The terminal of the first medical practitioner is a terminal used by the first medical practitioner, which includes various terminals such as a smartphone, a desktop computer, a laptop computer, and various smart wearable devices. The first medical practitioner interacts information with the information interaction platform by the terminal of the first medical practitioner.

The first registration information includes identity information of the first medical practitioner, and permission information applied by the first medical practitioner in the first institution.

In some exemplary embodiments, an account of the first medical practitioner is logged in the terminal of the first medical practitioner, and the information interaction platform confirms the identity of the first medical practitioner through the account.

The institution registers in the information interaction platform before step 401, and exemplarily, the institution registers in the information interaction platform by authentication information of the institution.

In step 402, verification is performed on the first registration information.

Upon acquiring the first registration information, the information interaction platform verifies the first registration information.

In some exemplary embodiments, the process of performing the verification by the information interaction platform includes the following.

1. The information interaction platform provides the first registration information to the first institution, and the first institution is configured to perform the verification on the first registration information.

That is, the information interaction platform provides the first registration information to the first institution, and the first institution performs the verification of the first registration information. Specifically, the information interaction platform transmits the first registration information to an institution control device of the first institution, which serves as a terminal for the first institution to interact information with the information interaction platform. Exemplarily, the institution control device is a terminal logged with an institution account of the first institution, and the institution control device includes various terminals such as a mobile phone, a desktop computer, a laptop computer, and various smart wearable devices.

2. The first registration information passes the verification in response to receiving verification pass information provided by the first institution.

The information interaction platform determines that the first registration information passes the verification after receiving the verification pass information provided by the first institution. Correspondingly, the information interaction platform, after receiving the verification failure information provided by the first institution, determines that the first registration information fails the verification and stops performing subsequent steps. Additionally, the information interaction platform feeds notification information that the registration information fails the verification to the terminal of the first medical practitioner.

In step 403, in response to the first registration information passing the verification, first role data of the first medical practitioner in the first institution is established.

After the first registration information passes the verification, the information interaction platform establishes the first role data of the first medical practitioner in the first institution in the information interaction platform. Here, the first role data of the first medical practitioner in the first institution refers to the first role data of the first medical practitioner corresponding to the first institution established by the information interaction platform, and in the method according to some embodiments of the present disclosure, the information interaction platform establishes the role data of any medical practitioner in an institution by steps 401 to 403.

In some embodiments of the present disclosure, the role data is configured to indicate various information about the medical practitioner in the institution, such as functions, available functions, data query ranges, and patients corresponding to the medical practitioner (e.g., patients for whom the medical practitioner is responsible). Exemplarily, the role data is a health manager or a doctor. The institution 1 and the institution 2 provide various services to the patients, such as acquiring patient description information of the patient through the corresponding terminal, providing service packages to the patients, or providing diagnosis services to the patients. The functions of the medical practitioners and the functions available to them in the different institutions are the same, but the data query ranges are the same or different.

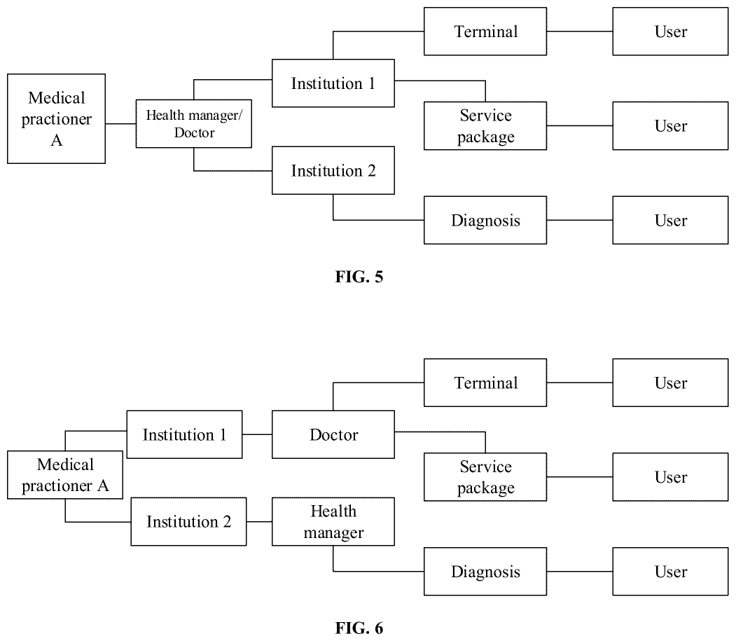

Exemplarily, as shown in FIG. 5, FIG. 5 is a schematic diagram of medical practitioners, institution, and role data according to some embodiments of the present disclosure.

The medical practitioner A has the same role data in the institution 1 and the institution 2 (which is a health manager or a doctor), and the institution 1 and the institution 2 provide various services to the patient, such as acquiring patient description information of the patient through the corresponding terminal, providing the patient with a service package (the service package includes a variety of services provided by the institution to the patient, such as health management and basic checkup), or providing diagnosis services to the patient. The functions of the medical practitioners and the functions available to them are the same in different institutions, but the data query ranges are the same or different.

In some embodiments of the present disclosure, the patient description information includes patient personal information (including patient name, gender, age, and the like), pre-questioning information (including body temperature, whether there is a part of the body that is uncomfortable, and if it is uncomfortable, how long the symptom exists, and other information about the physical condition), diagnosis results of the condition, pre-questioning information, a department that the patient wants to see, daily monitoring data (e.g., heart rate data, blood pressure data, and blood glucose data daily testing), and other information.

As shown in FIG. 6, FIG. 6 is another schematic diagram of medical practitioners, institution, and role data according to some embodiments of the present disclosure.

The medical practitioner A has different role data in the institution 1 and the institution 2. The role data in the institution 1 includes a doctor, and the role data in the institution 2 includes a health manager, whereby the functions of the medical practitioner A in different institutions and the functions available to the medical practitioner A are different, and the data query ranges are also different.

In step 404, second registration information provided by the terminal of the first medical practitioner for a second institution is acquired.

For the content of step 404, reference is made to step 401 described above.

In step 405, verification is performed on the second registration information.

For the content of step 405, reference is made to step 402 described above.

In step 406, in response to the second registration information passing the verification, second role data of the first medical practitioner in the second institution is established.

For the content of step 406, reference is made to step 403 described above.

The medical practitioner registers the same or different role data in different institutions through the above steps.

It should be noted that step 404, step 405, and step 406 are steps for the medical practitioner to register the role data in the second institution, and step 401, step 402, and step 403 are steps for the medical practitioner to register the role data in the first institution. The embodiments of the present disclosure do not restrict the sequence of the steps for registering the role data in the second institution and the steps for registering the role data in the first institution. Exemplarily, it is possible to perform step 404, step 405, and step 406 to register the role data in the second institution first, and then perform step 401, step 402, and step 403 to register the role data in the first institution.

Alternatively, the steps of registering the role data in the second institution and the steps of registering the role data in the first institution are performed simultaneously. Exemplarily, in one step, the information interaction platform acquires the first registration information provided for the first institution and the second registration information for the second institution by the terminal of the first medical practitioner. In another step, the information interaction platform verifies the first registration information and the second registration information, the specific verification process refers to step 402 described above, after which the information interaction platform establishes the role data based on the verification results.

It should be noted that in the embodiments of the present disclosure, the first institution involved refers to an institution registered on the information interaction platform, and the second institution refers to another institution registered on the information interaction platform.

In step 407, patient description information provided by the terminal corresponding to the first institution is acquired.

The terminal corresponding to the first institution is configured to acquire the patient description information provided by a target patient of the first institution, which is a patient of the first institution. Exemplarily, the terminal corresponding to the first institution refers to a terminal that is bound to the first institution, which belongs to the first institution or also belongs to the patient. The patient transmits the patient description information to the information interaction platform by the terminal corresponding to the first institution.

In step 408, role data of the plurality of medical practitioners corresponding to the first institution on the information interaction platform is acquired.

The information interaction platform determines the role data of the plurality of medical practitioners corresponding to the first institution on the information interaction platform, and the step of determining the role data is a step of searching and acquiring the role data by the information interaction platform. Exemplarily, after acquiring the patient description information provided by the terminal corresponding to the first institution in step 407, the information interaction platform starts searching and acquiring the role data of the plurality of medical practitioners corresponding to the first institution. Exemplarily, the role data of the plurality of medical practitioners corresponding to the first institution is stored in a data storage server, and the information interaction platform acquires the role data of the plurality of medical practitioners corresponding to the first institution from the data storage server.

In step 409, the patient description information is transmitted, based on the role data, to the terminal of at least one medical practitioner of the plurality of medical practitioners corresponding to the first institution.

After determining the role data of the plurality of medical practitioners corresponding to the first institution, the information interaction platform transmits the patient description information based on the role data to the terminal of at least one medical practitioner of the plurality of medical practitioners corresponding to the first institution.

The information interaction platform transmits the patient description information in a variety of ways.

Exemplarily, one way may include the following.

1. The information interaction platform determines, based on the role data, whether the target patient has a corresponding medical practitioner in the first institution.

The information interaction platform determines, based on the role data of the plurality of medical practitioners corresponding to the first institution, whether a corresponding medical practitioner exists for the target patient who provides the patient description information. Exemplarily, the first institution assigns a medical practitioner to the target patient prior to this step, and the assigned medical practitioner is the corresponding medical practitioner of the target patient in the first institution. Alternatively, the target patient actively establishes a correspondence with a medical practitioner in the first institution prior to this step (e.g., by making an appointment or registering with the medical practitioner at the first institution).

2. In a case where the target patient has the corresponding medical practitioner in the first institution, the information interaction platform transmits the patient description information to the terminal of the corresponding medical practitioners.

In the case where the target patient has the corresponding medical practitioner in the first institution, the information interaction platform transmits the patient description information to the terminal of the corresponding medical practitioner, such that the corresponding medical practitioner is capable of timely accessing the patient description information of the responsible patient.

3. In a case where the target patient does not have a corresponding medical practitioner in the first institution, the information interaction platform transmits the patient description information to the terminals of the corresponding plurality of medical practitioners in the first institution.

In the case where the target patient does not have the corresponding medical practitioners in the first institution, the information interaction platform transmits the patient description information to each of the medical practitioners corresponding to the first institution, and the patient description information is accessed by an idle medical practitioner.

After acquiring the patient description information, the medical practitioner acquires various conditions of the patient and makes further processing, such as contacting the target patient for further diagnosis or examination.

In step 410, a query request transmitted by the terminal of the first medical practitioner is acquired.

The information interaction platform provided by the embodiments of the present disclosure also provides a function of data query to the medical practitioners. Exemplarily, the information interaction platform acquires the query request transmitted by the terminal of the first medical practitioner, which is configured to acquire the patient description information of the target patient of the first institution.

It should be noted step 410 is performed at various times after acquiring the patient description information provided by the terminal corresponding to the first institution in step 407, which is not limited herein.

In step 411, whether the patient description information is within a query range of the first medical practitioner is determined based on the role data.

The information interaction platform determines, based on the role data, whether the patient description information queried by the first medical practitioner is within the query range of the first medical practitioner.

In step 412, in a case where the patient description information is within the query range of the first medical practitioner, the patient description information is transmitted to the terminal of the first medical practitioner.

The information interaction platform transmits the patient description information to the terminal of the first medical practitioner in the case where the patient description information is within the query range of the first medical practitioner.

Exemplarily, in a case where the role data of the first medical practitioner in the first institution is a health manager, the corresponding query range in the role data of the first medical practitioner includes patient description information related to health management, but in a case where the first medical practitioner requests a query for the patient description information that is not related to the health management, the patient description information is not within the query range of the first medical practitioner, and thus the information interaction platform does not transmit the patient description information to the terminal of the first medical practitioner.

In summary, according to the information interaction method provided by the embodiments of the present disclosure, by corresponding the patient to the institution in the information interaction platform, after acquiring the patient description information provided by the terminal corresponding to the first institution, the patient description information is transmitted to the terminal of the medical practitioners of the first institution based on the role data of the plurality of medical practitioners corresponding to the first institution. In this way, the information interaction platform is capable of transmitting the patient description information of the patients corresponding to each institution to the medical practitioners of the corresponding institution, such that the medical practitioners of different institutions cannot see the patient description information of other institutions, which improves the privacy of the patient description information and solves the problem of low protection of the privacy of patients in the related art, and thus the effect of improving the protection of the privacy of patients is achieved.

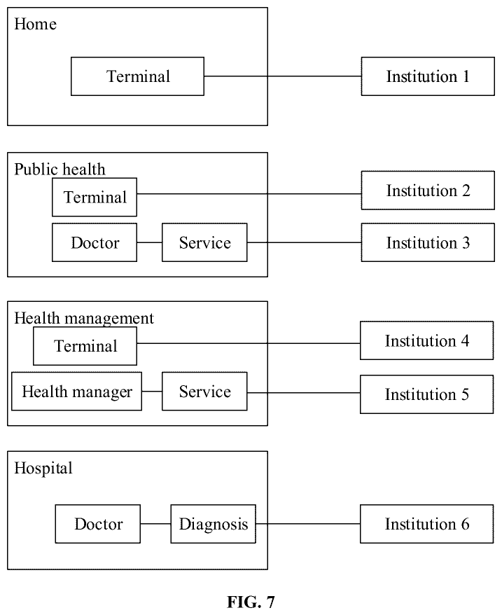

The information interaction method according to some embodiments of the present disclosure is applied in a variety of scenarios. Exemplarily, as shown in FIG. 7, FIG. 7 is a schematic diagram of some application scenarios of an information interaction method according to some embodiments of the present disclosure. These application scenarios include a home (or indoor) scenario, a public healthcare scenario, a healthcare management scenario, and a hospital scenario. The home scenario includes terminals corresponding to the institution, which are medical devices (e.g., a blood pressure meter, a blood glucose meter, and other health testing devices), and the institution 1 acquires the patient description information through these terminals. In the home scenario, the patient interacts information with the information interaction platform at home. In the public health institution scenario, terminals corresponding to the institution and doctors are included. The terminals and medical practitioners correspond to different institutions, and the doctor establishes an association with the institution by providing services. In the health management scenario, terminals and health managers are included. The health manager establishes an association with the institution by providing services. In the hospital scenario, the doctor provides diagnosis and treatment services to the corresponding institution for treating patients of the institution.

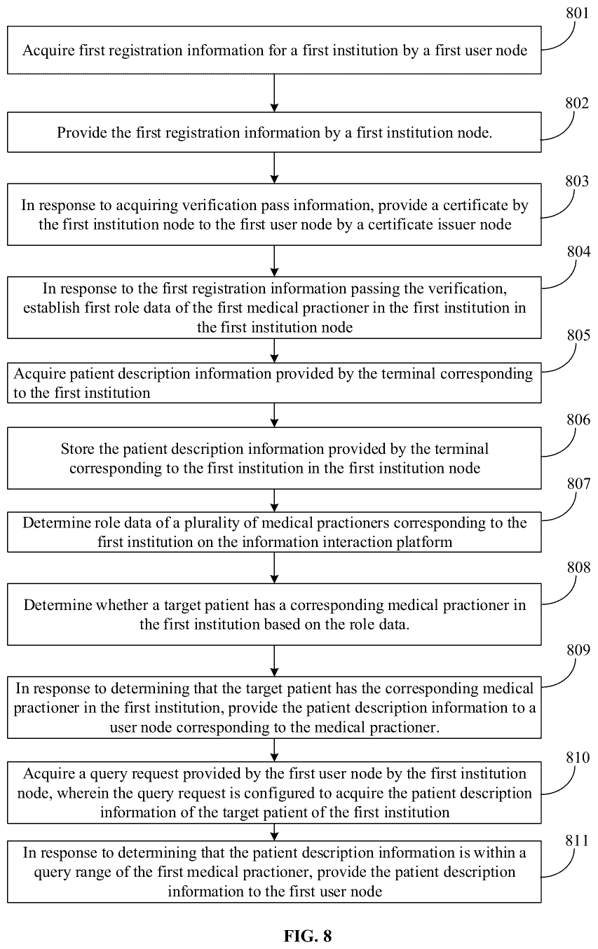

FIG. 8 is a flowchart of still another information interaction method according to some embodiments of the present disclosure. The method is used in the information interaction platform in the implementation environment shown in FIG. 2. The method includes the following steps.

In step 801, first registration information for a first institution is acquired by a first user node.

The first registration information includes identity information of a first medical practitioner, and permission information applied by the first medical practitioner in the first institution

The information interaction method according to some embodiments of the present disclosure is applied to an information interaction platform as a block chain platform. In this case, the block chain platform acquires the first registration information for the first institution by the first user node. The first user node is a node that establishes a connection with a terminal of the first medical practitioner, or the terminal of the first medical practitioner becomes such a first user node after accessing the block chain. It should be noted that, in the embodiment of the present disclosure, the first medical practitioner registers a personal digital identity in the information interaction platform, which is bound to an account that the first medical practitioner logs into in the terminal, and the registration information of various institutions registered by the first medical practitioner thereafter is bound to the personal digital identity without the need to register in the various institutions through different accounts.

Role data is registered in each institution by the first user node in the information interaction platform

A block chain is a linked data structure in which data blocks are assembled sequentially chronologically, and is a distributed ledger cryptographically guaranteed to be tamper-proof and unforgeable. Broadly speaking, the block chain technology is a new distributed infrastructure and computing method that verifies and stores data using a block-chain data structure, generates and updates data using distributed node consensus algorithms, ensures the security of data transmission and access using cryptographic methods, and programs and manipulates data using smart contracts consisting of automated script code. The block chain is essentially thought of as a decentralized database.

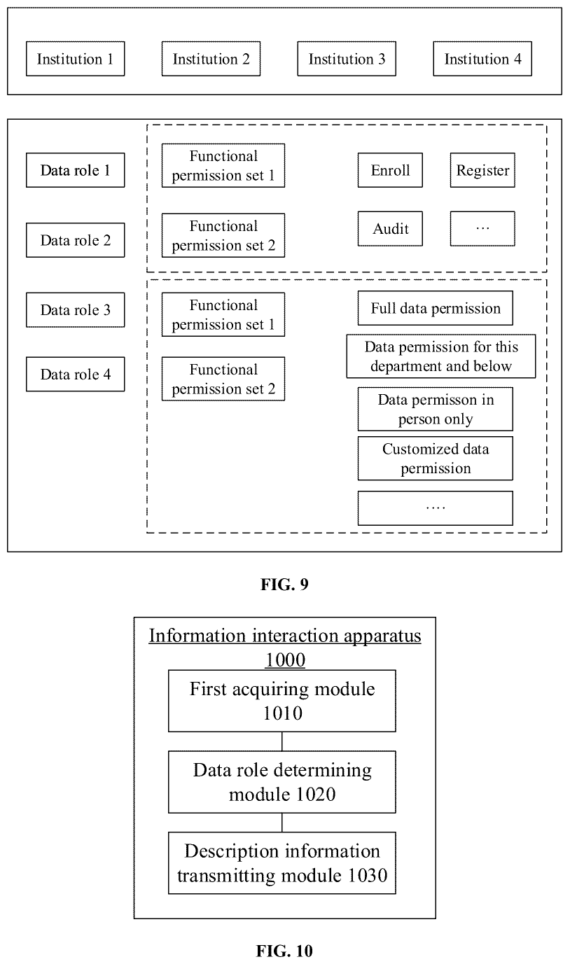

Further, as shown in FIG. 9, FIG. 9 is a schematic diagram of permission management according to some embodiments of the present disclosure. Institutions registered on the information interaction platform include an institution 1, an institution 2, an institution 3, and an institution 4, and each institution includes role data 1, role data 2, role data 3, and role data 4. Each role data includes a functional permission set and a data permission set. The functional permission set includes a functional permission set 1 and a functional permission set 2, and the functional permission set 1 and the functional permission set 2 include at least one of various functions such as registration, enrolment, auditing, and the like. The data permission set includes a data permission set 1 and a data permission set 2, and the data permission set 1 and the data permission set 2 include any one of all data permission, data permission in this department and below, data permission in person only, self-defined data permission, or other data permission. Exemplarily, the permission in this department indicates that only data related to the present department can be seen; the all data permission indicates that all data in the institution can be seen; the self-defined data permission indicates that the department or other customization can be selected; the data permission in this department and below indicates that the data of the present department and its subordinate sub-departments can be seen; and the data permission in person only indicates that only data generated by and related to oneself can be seen.

In step 802, the first registration information is provided by a first institution node.

The block chain platform provides the first registration information acquired in step 801 to the first institution node. Exemplarily, the first registration information is transmitted directly by the first user node to the first institution node. The first institution node is a node assigned to the first institution, and various data related to the first institution is stored in the first institution node, which enhances the data security and privacy

The first institution acquires the first registration information from the first institution node and verifies the first registration information to verify the identity authentication of the first medical practitioner. In a case where the first registration information is authentic and valid information, the first institution provides verification pass information to the first institution node, and correspondingly, in a case where the first registration information is not authentic and valid information, the first institution provides verification failure information to the first institution node.

In step 803, in response to acquiring verification pass information, the first institution node provides a certificate to the first user node by a certificate issuer node.

The information interaction platform provides the certificate to the first user node by the certificate issuer node when the first institution node acquires the verification pass information. The certificate issuer node is a node for issuing certificates to various nodes in the block chain. The certificate is configured to determine that the first registration information passes the verification. Exemplarily, the certificate includes a registration certificate (representing the identity of the medical practitioner) and a transaction certificate (representing the permission of the medical practitioner, e.g., the medical practitioner A has permission to view the patient description information in the first institution).

In step 804, in response to the first registration information passing the verification, first role data of the first medical practitioner in the first institution is established in the first institution node.

The information interaction platform establishes the first role data of the first medical practitioner in the first institution in the first institution node in the case where the first registration information passes the verification. Since this first role data is stored in the first institution node corresponding to the first institution, the security and privacy of the data are high.

Step 801 to step 804 are the process of registering the first role data in the first institution by the first medical practitioner through the first user node, and the first medical practitioner also registers the second role data in the second institution through the first user node, and similarly, the second role data is stored in the second institution node.

In step 805, patient description information provided by the terminal corresponding to the first institution is acquired.

The information interaction platform acquires the patient description information provided by the terminal corresponding to the first institution. In some exemplary embodiments, the terminal corresponding to the first institution is accessed in the block chain to be a target patient node, and the target patient provides the patient description information to the target patient node. Specific contents of the patient description information are referred to the above embodiments, which are not repeated herein.

In step 806, the patient description information provided by the terminal corresponding to the first institution is stored in the first institution node.

The information interaction platform stores the patient description information provided by the target patient in the first institution node, and other institution nodes or practitioners unrelated to the first institution fail to access the patient description information, such that the security and privacy of the patient description information are ensured.

Similarly, the information interaction platform acquires patient description information provided by the terminal corresponding to the second institution, and stores the patient description information provided by the terminal corresponding to the second institution in the second institution node.

In step 807, role data of a plurality of medical practitioners corresponding to the first institution on the information interaction platform is acquired.

The information interaction platform determines the role data of the plurality of medical practitioners corresponding to the first institution on the information interaction platform. The step of determining the role data is a step that the information interaction platform searching and acquiring the role data. Exemplarily, the role data of the plurality of medical practitioners corresponding to the first institution is stored in the first institution node, and then the role data of the first institution corresponding to the first institution is acquired by the first institution node.

For the specific description of the role data, reference is made to the above embodiments, which is not repeated herein.

In step 808, whether a target patient has a corresponding medical practitioner in the first institution is determined based on the role data.

The first institution node in the information interaction platform determines, based on the role data of the plurality of medical practitioners corresponding to the first institution, whether the corresponding medical practitioners is present for the target patient who provides the patient description information. Exemplarily, the first institution has a medical practitioner assigned to the target patient prior to this step, and then the assigned medical practitioner is the corresponding medical practitioner of the target patient in the first institution. Alternatively, the target patient actively establishes a correspondence with a medical practitioner in the first institution prior to this step (e.g., by making an appointment or registering with the medical practitioner at the first institution).

In step 809, in a case where the target patient has the corresponding medical practitioner in the first institution, the patient description information is provided to a user node corresponding to the medical practitioner.

The first institution node provides the patient description information to the user node corresponding to the medical practitioner when determining that the target patient has the corresponding medical practitioner in the first institution. The user node corresponding to that medical practitioner is a node used by the medical practitioners for information interaction with other nodes in the information interaction platform. After providing the patient description information to the user node, the medical practitioner corresponding to the target patient in the first institution accesses the patient description information of the target patient.

Alternatively, in a case where the target patient does not have the corresponding medical practitioners in the first institution, the information interaction platform transmits the patient description information to the user node corresponding to respective medical practitioners corresponding to the first institution, and the patient description information is accessed by an idle medical practitioner.

After acquiring the patient description information, the medical practitioner acquires various conditions of the patient and makes further processing, such as contacting the target patient for further diagnosis or examination.

In step 810, a query request provided by the first user node is acquired by the first institution node, wherein the query request is configured to acquire the patient description information of the target patient of the first institution.

The information interaction platform acquires, by the first institution node, the query request provided by the first user node, which is configured to acquire the patient description information of the target patient of the first institution. Exemplarily, the information interaction platform acquires the query request provided by the first user node after storing the patient description information provided by the terminal corresponding to the first institution in the first institution node (after step 806).

In some exemplary embodiments, step 810 includes the following.

1. A query request transmitted by a node to be verified is received by the first institution node.

The query request is configured to request the patient description information of the target patient of the first institution, then the information interaction platform receives the query request transmitted by the to-be-verified node by the first institution node. The identity of the to-be-verified node is a node that has not been authenticated, and the information interaction platform has not yet been informed whether the to-be-verified node is a node corresponding to the medical practitioner of the first medical institution, such that the information interaction platform acquires the query request for the institution through the corresponding institution node, which enhances the security of the data and the privacy.

2. The identity of the node to be verified is authenticated.

After acquiring the query request transmitted by the to-be-verified node, the information interaction platform starts to perform identity authentication on the to-be-verified node. In performing the identity authentication, based on an institution to which the query request is directed, the identity authentication is performed by an institution node of the institution. Exemplarily, the identity authentication is performed by the first institution node that receives the query request in the embodiments of the present disclosure, and the first institution node, in performing the identity authentication on the to-be-verified node, verifies a certificate of the to-be-verified node, which is the certificate that is issued to the user node by the certificate issuer node in step 803 and is included in the query request; alternatively, the certificate is requested by the first institution node to the to-be-verified node; alternatively, the certificate is transmitted by the to-be-verified node to the to-be-verified node.

3. In a case where the to-be-verified node passes the identity authentication, the to-be-verified node is determined to be the first user node.

The information interaction platform determines the to-be-verified node to be the first user node in response to the to-be-verified node passing the identity authentication. Specifically, the first institution node determines that the to-be-verified node is the first user node.

In some embodiments, the information interaction platform also determines whether the to-be-verified node is the first user node by other nodes, such as determining whether the to-be-verified node is the first user node by the certificate issuer node.

In step 811, in a case where the patient description information is within a query range of the first medical practitioner, the patient description information is provided to the first user node.

The first institution node in the information interaction platform provides the patient description information to the first user node in the case where the patient description information is within the query range of the first medical practitioner.

Alternatively, the first institution node in the information interaction platform does not provide the patient description information to the first user node in a case where the patient description information is not within the query range of the first medical practitioner and provides feedback to the first user node with notification information that the query is out of range.

In summary, according to the information interaction method provided by the embodiments of the present disclosure, by corresponding the patient to the institution in the information interaction platform, after acquiring the patient description information provided by the terminal corresponding to the first institution, the patient description information is transmitted to the terminal of the medical practitioner of the first institution based on the role data of the plurality of medical practitioners corresponding to the first institution. In this way, the information interaction platform is capable of transmitting the patient description information of the patients corresponding to each institution to the medical practitioners of the corresponding institution, such that the medical practitioners of different institutions cannot see the patient description information of other institutions, which improves the privacy of the patient description information and solves the problem of low protection of the privacy of the patients in the related art, and thus the effect of improving the protection of the privacy of the patients is achieved.

The following are apparatus embodiments of the present disclosure that are applicable to performing the method embodiments described above. For details not disclosed in the apparatus embodiments of the present disclosure, references are made to the method embodiments of the present disclosure.

FIG. 10 is a block diagram of an information interaction apparatus according to some embodiments of the present disclosure. The information interaction apparatus is implemented as part or all of an information interaction platform through software, hardware, or a combination of both. The information interaction device 1000 includes:
a first acquiring module 1010, configured to acquire patient description information provided by a terminal corresponding to a first institution, wherein the first institution is an institution registered on the information interaction platform, and the terminal corresponding to the first institution is configured to acquire patient description information provided by a target patient of the first institution;
a role data determining module 1020, configured to determine role data of a plurality of medical practitioners corresponding to the first institution on the information interaction platform, wherein the role data is configured to identify information of the medical practitioners in the first institution; and
a description information transmitting module 1030, configured to transmit, based on the role data, the patient description information to a terminal of at least one medical practitioner of the plurality of medical practitioners corresponding to the first institution.

In some embodiments, the information interaction apparatus further includes:
a first registering module, configured to acquire first registration information provided by the terminal of the first medical practitioner for the first institution;
a first verifying module, configured to perform verification on the first registration information;
a first establishing module, configured to establish first role data of the first medical practitioner in the first institution in response to the first registration information passing the verification;
a second registering module, configured to acquire second registration information provided by the terminal of the first medical practitioner for a second institution;
a second verifying module, configured to perform verification on the second registration information; and
a second establishing module, configured to establish second role data of the first medical practitioner in the second institution when the second registration information passes the verification.

In some embodiments, the first verifying module includes:
a first transmitting unit, configured to transmit the first registration information to a verification terminal, wherein the verification terminal is configured to perform verification on the first registration information; and
a first determining unit, configured to determine that the first registration information passes the verification in response to receiving verification pass information provided by the verification terminal.

In some embodiments, the information interaction platform further includes a certificate issuer node, a first user node, and a first institution node corresponding to the first institution in a block chain. The first user node includes a terminal of the first medical practitioner corresponding to the first institution, and the verification terminal includes the first institution node.

The first registration module is further configured to:
acquire first registration information provided by the first user node for the first institution.

The first determining unit is further configured to:
provide a certificate to the first user node by the certificate issuer node in response to receiving the verification pass information provided by the first institution node, wherein the certificate is configured to determine that the first registration information passes the verification.

In some embodiments, after acquiring the patient description information provided by the terminal corresponding to the first institution, the information interaction unit further includes:
a first storing module, configured to store the patient description information provided by the terminal corresponding to the first institution in the first institution node;
a second acquiring module, configured to acquire the patient description information provided by the terminal corresponding to the second institution; and
a second storing module, configured to store the patient description information provided by the terminal corresponding to the second institution in the second institution node.

In some embodiments, the role data incudes a data query range of the medical practitioners in the first institution, and the information interaction apparatus further includes:
a request acquiring module, configured to acquire a query request transmitted by the first user node, wherein the query request is configured to acquire the patient description information of the target patient in the first institution;
a range determining module, configured to determine, based on the role data, whether the patient description information is within the query range of the first medical practitioner; and
a query information transmitting module, configured to transmit the patient description information to the first user node in response to determining that the patient description information is within the query range of the first medical practitioner.

In some embodiments, the request acquiring module includes:
a request receiving unit, configured to receive a query request transmitted by a to-be-verified node by the first institution node;
a verifying unit, configured to perform identity authentication on the node to-be-verified; and
a determining unit, configured to determine, in response to the to-be-verified node passing the identity authentication, that the to-be-verified node is the first user node.

In some embodiments, the verifying unit is further configured to:
perform verification on a certificate provided by the to-be-verified node; and
determine that the to-be-verified node passes the identity authentication in response to the certificate provided by the to-be-verified node passing the verification.

In some embodiments, the description information transmitting module is further configured to:
determine, based on the role data, whether the target patient has a corresponding medical practitioner in the first institution; and
in response to the target patient having the corresponding medical practitioner in the first institution, transmit the patient description information to a terminal of the corresponding medical practitioner.

In some embodiments, the information interaction apparatus further includes:
an authorizing module, configured to acquire authorization information provided by the target patient, wherein the authorization information is configured to authorize the information interaction platform, such that the information interaction platform is capable of transmitting the patient description information to the terminal of at least one of the plurality of medical practitioners corresponding to the first institution.

In summary, according to the information interaction method provided by the embodiments of the present disclosure, by corresponding the patient to the institution in the information interaction platform, after acquiring the patient description information provided by the terminal corresponding to the first institution, the patient description information is transmitted to the terminal of the medical practitioner of the first institution based on the role data of the plurality of medical practitioners corresponding to the first institution. In this way, the information interaction platform is capable of transmitting the patient description information of the patients corresponding to each institution to the medical practitioners of the corresponding institution, such that the medical practitioners of different institutions cannot see the patient description information of other institutions, which improves the privacy of the patient description information and solves the problem of low protection of the privacy of the patients in the related art, and thus the effect of improving the protection of the privacy of the patients is achieved.

Some embodiments of the present disclosure further provide an information interaction device. The information interaction device includes a processor and a memory. The memory stores at least one instruction, at least one program, a code set, or an instruction set therein. The at least one instruction, the at least one program, the code set, or the instruction set are loaded and executed by the processor to perform an information interaction method. The method includes:
acquiring patient description information provided by a terminal corresponding to a first institution, wherein the first institution is an institution registered on the information interaction platform, and the terminal corresponding to the first institution is configured to acquire the patient description information provided by a target patient of the first institution;
determining role data of a plurality of medical practitioners corresponding to the first institution on the information interaction platform, wherein the role data is configured to identify information of the medical practitioners in the first institution; and
transmitting, based on the role data, the patient description information to a terminal of at least one medical practitioner of the plurality of medical practitioners corresponding to the first institution.

Some embodiments of the present disclosure further provide a non-transitory computer storage medium. The computer storage medium stores therein at least one instruction, at least one program, a code set, or an instruction set. The at least one instruction, the at least one program, the code set, or the instruction set is loaded and executed by a processor to perform an information interaction method as described above.

Further, some embodiments of the present disclosure provide an information interaction platform. The information interaction platform is configured to:
acquire patient description information provided by a terminal corresponding to a first institution, wherein the first institution is an institution registered on the information interaction platform, and the terminal corresponding to the first institution is configured to acquire the patient description information provided by a target patient of the first institution;
determine role data of a plurality of medical practitioners corresponding to the first institution on the information interaction platform, wherein the role data is configured to identify information of the medical practitioners in the first institution; and
transmit, based on the role data, the patient description information to a terminal of at least one medical practitioner of the plurality of medical practitioners corresponding to the first institution.

In some embodiments, the information interaction platform includes the terminal of the first medical practitioner, and the terminal of the first medical practitioner is configured to acquire first registration information for the first institution.

The information interaction platform is configured to:
perform verification on the first registration information; and
in a case where the first registration information passes the verification, establish first role data of the first medical practitioner in the first institution.

The terminal of the first medical practitioner is further configured to acquire second registration information for the second institution.

The information interaction platform is further configured to:
perform verification on the second registration information; and
in a case where the second registration information passes the verification, establish second role data of the first medical practitioner in the second institution.

In some embodiments, the information interaction platform further includes a certificate issuer node in the block chain, a first user node, and a first institution node corresponding to the first institution. The first user node includes the terminal of the first medical practitioner corresponding to the first institution.

The first user node is configured to provide the first registration information for the first institution.

The first institution node is configured to perform verification on the first registration information.

The certificate issuer node is configured to provide a certificate to the first user node in a case where the first registration passes the verification, and the certificate is configured to determine that the first registration information passes the verification.

In addition, other contents of the information interaction platform are referred to other embodiments of the present disclosure, which are not repeater herein.

According to another aspect of some embodiments of the present disclosure, a computer program product or a computer program is provided. The computer program product or computer program includes one or more computer instructions. The one or more computer instructions are stored in a computer-readable storage medium. The one or more computer instructions, when read and loaded by a processor of the computer device from the computer-readable storage medium, cause the processor of the computer device to perform the methods provided in the various embodiments described above.

The term “at least one of A and B” mentioned in the present disclosure is merely a description of an association relationship of an associated object, indicating that three types of relationships may exist, e.g., at least one of A and B means the following three cases: A exists alone, A and B exist at the same time, and B exits alone. Similarly, “at least one of A, B, and C” means that seven relationships: A exists alone, B exists alone, C exists alone, both A and B exist, both A and C exist, both C and B exist, and A, B, and C exist. Similarly, “at least one of A, B, C and D” means fifteen relationships: A exists alone, B exists alone, C exists alone, D exists alone, both A and B exist, both A and C exist, both A and D exist, both C and B exist, both D and B exist, both C and D exist, A, B, and C exist at the same time, A, B, and D exist at the same time, A, C, and D exist at the same time, B, C, and D exist at the same time, A, B, C, and D exist at the same time.

In the present disclosure, the terms “first” and “second” are only used for descriptive purposes, and cannot be understood as indicating or implying relative importance. The term “a plurality of” refers to two or more, unless expressly defined otherwise.

It should be noted that the information (including, but not limited to, patient description information, personal information of the patient, and the like.), data (including, but not limited to, data used for analysis, stored data, displayed data, and the like.), and signals involved in the present disclosure are authorized by the patient or sufficiently authorized by the parties. The collection, use, and processing of the relevant data are subject to relevant laws, regulations, and standards of the relevant countries and regions. laws regulations and standards. For example, the patient descriptive information involved in this application is acquired with full authorization.

In the several embodiments provided in this application, it should be understood that the apparatus and methods disclosed, may be implemented in other ways. For example, the above-described embodiments of the apparatus are merely schematic. For example, the division of the units described is merely a logical functional division, and the actual implementation may be divided in other ways, e.g., a plurality of units or components may be combined or may be integrated into another system, or some features may be ignored, or not implemented. As another point, the coupling or direct coupling or communication connection between each other shown or discussed may be an indirect coupling or communication connection through some interface, device, or unit, which may be electrical, mechanical, or otherwise.

The units illustrated as separated components may or may not be physically separated, and components shown as units may or may not be physical units, i.e., they may be located in a single place or they may be distributed to a plurality of network units. Some or all of these units may be selected to achieve the purpose of the present embodiment according to actual needs.

Those skilled in the art may understand that all or some of the steps for realizing the above embodiments may be done by hardware, or may be done by a program to instruct the relevant hardware to do so and that the program may be stored in a computer-readable storage medium, and that the storage medium referred to above may be a read-only memory, a magnetic disc or a compact disc, and the like.

Described above are merely exemplary embodiments of the present disclosure, and are not intended to limit the present disclosure. Therefore, any modifications, equivalent substitutions, improvements, and the like made within the spirit and principles of the present disclosure shall be included in the protection scope of the present disclosure.

1. An information interaction method, applicable to an information interaction platform, the method comprising:
acquiring patient description information provided by a terminal corresponding to a first institution, wherein the first institution is an institution registered on the information interaction platform, and the terminal corresponding to the first institution is configured to acquire the patient description information provided by a target patient of the first institution;
determining role data of a plurality of medical practitioners corresponding to the first institution on the information interaction platform, wherein the role data is configured to identify information of the medical practitioners in the first institution; and
transmitting, based on the role data, the patient description information to a terminal of at least one medical practitioner of the plurality of medical practitioners corresponding to the first institution.

2. The method according to claim 1, wherein prior to acquiring the patient description information provided by the terminal corresponding to the first institution, the method further comprises:
acquiring first registration information provided by a terminal of a first medical practitioner for the first institution;
performing verification on the first registration information;
in response to the first registration information passing the verification, establishing first role data of the first medical practitioner in the first institution;
acquiring second registration information provided by the terminal of the first medical practitioner for a second institution;
performing verification on the second registration information; and
in response to the second registration information passing the verification, establishing second role data of the first medical practitioner in the second institution.

3. The method according to claim 2, wherein performing the verification on the first registration information comprises:
providing the first registration information to the first institution, wherein the first institution is configured to perform the verification on the first registration information; and
in response to receiving verification pass information provided by the first institution, determining that the first registration information passes the verification.

4. The method according to claim 3, wherein
the information interaction platform further comprises: a certificate issuer node, a first user node, and a first institution node corresponding to the first institution in a block chain; and
acquiring the first registration information provided by the terminal of the first medical practitioner for the first institution comprises:
acquiring, by the first user node, the first registration information provided by the terminal of the first medical practitioner for the first institution;
providing the first registration information to the first institution comprises:
providing the first registration information to the first institution node; and
in response to receiving the verification pass information provided by the first institution, determining that the first registration information passes the verification comprises:
in response to acquiring the verification pass information, providing a certificate to the first user node by the certificate issuer node, wherein the certificate is configured to determine that the first registration information passes the verification.

5. The method according to claim 4, wherein upon acquiring the patient description information provided by the terminal corresponding to the first institution, the method further comprises:
storing the patient description information provided by the terminal corresponding to the first institution in the first institution node;
acquiring patient description information provided by a terminal corresponding to the second institution; and
storing the patient description information provided by the terminal corresponding to the second institution in the second institution node.

6. The method according to claim 4, wherein
the role data comprises a data query range of the medical practitioners in the first institution; and
the method further comprises:
acquiring, by the first institution node, a query request provided by the first user node, wherein the query request is configured to acquire the patient description information of the target patient of the first institution, and the first institution node is configured to determine, based on the role data, whether the patient description information is within a query range of the first medical practitioner; and
in response to determining that the patient description information is within the query range of the first medical practitioner, providing the patient description information to the first user node.

7. The method according to claim 6, wherein acquiring, by the first institution node, the query request provided by the first user node comprises:
receiving, by the first institution node, the query request transmitted by a to-be-verified node;
performing identity authentication on the to-be-verified node; and
in response to the to-be-verified node passing the identity authentication, determining the to-be-verified node to be the first user node.

8. The method according to claim 7, wherein performing the identity authentication on the to-be-verified node comprises:
performing verification, by the first institution node, on a certificate provided by the to-be-verified node;
in response to the certificate provided by the to-be-verified node passing the verification, determining that the to-be-verified node passes the identity authentication.

9. The method according to claim 1, wherein transmitting, based on the role data, the patient description information to the terminal of at least one medical practitioner of the plurality of medical practitioners corresponding to the first institution comprises:
determining, based on the role data, whether the target patient has a corresponding medical practitioner in the first institution; and
in response to the target patient having the corresponding medical practitioner in the first institution, transmitting the patient description information to a terminal of the corresponding medical practitioners.

10. The method according to claim 1, wherein prior to transmitting, based on the role data, the patient description information to the terminal of at least one medical practitioner of the plurality of medical practitioners corresponding to the first institution, the method further comprises:
acquiring authorization information provided by the target patient, wherein the authorization information is configured to authorize the information interaction platform, such that the information interaction platform is capable of transmitting the patient description information to the terminal of at least one medical practitioner of the plurality of medical practitioners corresponding to the first institution.

11. An information interaction platform, configured to:
acquire patient description information provided by a terminal corresponding to a first institution, wherein the first institution is an institution registered on the information interaction platform, and the terminal corresponding to the first institution is configured to acquire the patient description information provided by a target patient of the first institution;
determine role data of a plurality of medical practitioners corresponding to the first institution on the information interaction platform, wherein the role data is configured to identify information of the medical practitioners in the first institution; and
transmit, based on the role data, the patient description information to a terminal of at least one medical practitioner of the plurality of medical practitioners corresponding to the first institution.

12. The information interaction platform according to claim 11, comprising: a terminal of a first medical practitioner, wherein the terminal of the first medical practitioner is configured to acquire first registration information for the first institution; and
the information interaction platform is further configured to:
perform verification on the first registration information; and
in response to the first registration information passing the verification, establish first role data of the first medical practitioner in the first institution;
the terminal of the first medical practitioner is further configured to acquire second registration information for a second institution; and
the information interaction platform is further configured to:
perform verification on the second registration information; and
in response to the second registration information passes the verification, establish second role data of the first medical practitioner in the second institution.

13. An information interaction device, comprising: a processor and a memory, the memory storing at least one instruction, at least one program, a code set, or an instruction set therein, wherein the processor, when loading and executing the at least one instruction, the at least one program, the code set, or the instruction set, is caused to perform:
acquiring patient description information provided by a terminal corresponding to a first institution, wherein the first institution is an institution registered on the information interaction platform, and the terminal corresponding to the first institution is configured to acquire the patient description information provided by a target patient of the first institution;
determining role data of a plurality of medical practitioners corresponding to the first institution on the information interaction platform, wherein the role data is configured to identify information of the medical practitioners in the first institution; and
transmitting, based on the role data, the patient description information to a terminal of at least one medical practitioner of the plurality of medical practitioners corresponding to the first institution.

14. The information interaction device according to claim 13, wherein the processor, when loading and executing the at least one instruction, the at least one program, the code set, or the instruction set, is caused to perform:
acquiring first registration information provided by a terminal of a first medical practitioner for the first institution;
performing verification on the first registration information;
in response to the first registration information passing the verification, establishing first role data of the first medical practitioner in the first institution;
acquiring second registration information provided by the terminal of the first medical practitioner for a second institution;
performing verification on the second registration information; and
in response to the second registration information passing the verification, establishing second role data of the first medical practitioner in the second institution.

15. The information interaction device according to claim 14, wherein the processor, when loading and executing the at least one instruction, the at least one program, the code set, or the instruction set, is caused to perform:
providing the first registration information to the first institution, wherein the first institution is configured to perform the verification on the first registration information; and
in response to receiving verification pass information provided by the first institution, determining that the first registration information passes the verification.

16. The information interaction device according to claim 15,
the information interaction platform further comprises a certificate issuer node, a first user node, and a first institution node corresponding to the first institution in a block chain; and
the processor, when loading and executing the at least one instruction, the at least one program, the code set, or the instruction set, is caused to perform:
acquiring, by the first user node, the first registration information provided by the terminal of the first medical practitioner for the first institution;
providing the first registration information to the first institution node; and
in response to acquiring the verification pass information, providing a certificate to the first user node by the certificate issuer node, wherein the certificate is configured to determine that the first registration information passes the verification.

17. The information interaction device according to claim 16, wherein the processor, when loading and executing the at least one instruction, the at least one program, the code set, or the instruction set, is caused to perform:
storing the patient description information provided by the terminal corresponding to the first institution in the first institution node;
acquiring patient description information provided by a terminal corresponding to the second institution; and
storing the patient description information provided by the terminal corresponding to the second institution in the second institution node.

18. The information interaction device according to claim 16, wherein
the role data comprises a data query range of the medical practitioners in the first institution; and
the processor, when loading and executing the at least one instruction, the at least one program, the code set, or the instruction set, is caused to perform:
acquiring, by the first institution node, a query request provided by the first user node, wherein the query request is configured to acquire the patient description information of the target patient of the first institution, and the first institution node is configured to determine, based on the role data, whether the patient description information is within a query range of the first medical practitioner; and
in response to determining that the patient description information is within the query range of the first medical practitioner, providing the patient description information to the first user node.

19. The information interaction device according to claim 18, wherein the processor, when loading and executing the at least one instruction, the at least one program, the code set, or the instruction set, is caused to perform:
receiving, by the first institution node, the query request transmitted by a to-be-verified node;
performing identity authentication on the to-be-verified node; and
in response to the to-be-verified node passing the identity authentication, determining the to-be-verified node to be the first user node.

20. A non-transitory computer storage medium, storing at least one instruction, at least one program, a code set, or an instruction set therein, wherein the at least one instruction, the at least one program, the code set, or the instruction set, when loaded and executed by a processor, causes the processor to perform the information interaction method as defined in claim 1.
